(19) United States
(12) Patent Application Publication
(10) Pub. No.: US 2024/0202913 A1
(43) Pub. Date: Jun. 20, 2024

(54) Calcium Arc Computation Relative to Lumen Center

(71) Applicant: LightLab Imaging, Inc., Westford, MA (US)

(72) Inventors: Timothy Preston Connelly, Medford, MA (US); Christopher Erik Griffin, Wilton, NH (US); Shimin Li, Acton, MA (US)

(73) Assignee: LightLab Imaging, Inc., Westford, MA (US)

(21) Appl. No.: 18/287,252

(22) PCT Filed: Apr. 22, 2022
(86) PCT No.: PCT/US2022/025961

§ 371 Date: Oct. 17, 2023

Related U.S. Application Data
(60) Provisional application No. 63/178,388, filed on Apr. 22, 2021.

(51) Int. Cl. G06T 7/00; A61B 5/02; G06T 5/20; G06T 5/50; G06T 5/70; G06T 7/11; G06T 7/13; G06T 7/66

(52) U.S. Cl. CPC G06T 7/0012; A61B 5/02007; G06T 5/20; G06T 5/50; G06T 5/70; G06T 7/11; G06T 7/13; G06T 7/66; G06T 2207/30101

(57) ABSTRACT

Methods, systems, and apparatus, including computer-readable storage media for calculating lumen-centered calcium arcs. A method includes receiving, by one or more processors, an image frame and an identification of a region of plaque in the image frame. The image frame is taken while the imaging device is in a lumen depicted in the image frame. The one or more processors identify a lumen-center of the lumen in the image frame, and generate a lumen-centered are having a coverage angle centered on the lumen-center, using at least the lumen-center.

### CROSS-REFERENCE TO RELATED APPLICATIONS

The present application claims the benefit of the filing date of U.S. Provisional Application No. 63/178,388, filed Apr. 22, 2021, entitled “CALCIUM ARC COMPUTATION RELATIVE TO LUMEN CENTER,” the disclosure of which is hereby incorporated herein by reference.

### BACKGROUND

Optical coherence tomography (OCT) is an imaging technique with widespread applications in ophthalmology, cardiology, gastroenterology, and other fields of medicine and scientific study. OCT can be used in conjunction with various other imaging technologies, such as intravascular ultrasound (IVUS), angiography, fluoroscopy, and X-ray based imaging.

To perform imaging, an imaging probe can be mounted on a catheter and maneuvered through a point of interest, such as a blood vessel of a patient. The imaging probe can return multiple image frames of the point of interest, which can be further processed or analyzed, for example to diagnose the patient with a medical condition, or as part of a scientific study. Normal arteries have a layered structure that includes intima, media, and adventitia. As a result of some medical conditions, such as atherosclerosis, the intima or other parts of the artery may contain plaque, which can be formed from different types of fiber, proteoglycans, lipid, or calcium.

### BRIEF SUMMARY

Aspects of the disclosure provide for computing coverage angles for arcs corresponding to regions of plaque in tissue around a lumen. A system configured as described herein can receive image data including an identification of plaque, such as calcium, in tissue around a lumen. The system can compute a coverage angle of a device-centered arc relative to the position of a device in the lumen at the time the device captured the image. The system can compute the center of the lumen and, using the lumen-center, generate a lumen-centered arc and a coverage angle corresponding to the arc. The processed image can be annotated with the lumen-centered arc, which can be further used as part of analyzing the image, for example as part of evaluating plaque in the image under a calcium scoring rubric.

In addition to providing image frames with lumen-centered arcs for calcium scoring, lumen-centered arcs can be displayed through a display viewport with less variation from frame-to-frame as compared with device-centered arcs. Device-centered arcs can vary greatly from frame-to-frame of the same lumen during a pullback, at least because the position of an imaging probe for an imaging device can vary within the lumen as the imaging device is maneuvered. In addition, depending on the field-of-view (FOV) set for viewing an image frame, device-centered arcs can appear larger or smaller than a corresponding detected region of plaque corresponding to the arc. Lumen-centered arcs calculated as described herein can be displayed without the aforementioned erratic variation caused by different FOVs and/or changes in position of the imaging device from frame-to-frame.

Aspects of the disclosure provide for a method including: receiving, by one or more processors, an image frame and an identification of a region of plaque in the image frame, wherein the image frame is taken by an imaging device while the imaging device was in a lumen depicted in the image frame; identifying, by the one or more processors, a lumen-center of the lumen depicted in the image frame; and generating, by the one or more processors and using at least the lumen-center, a lumen-centered arc having a coverage angle centered on the lumen-center.

The identification of the region of plaque can include a mask, wherein a region of pixels in the mask corresponds to the region of plaque depicted in the image frame.

The method can further include identifying endpoints of the region of plaque through which lines tangential to the region of plaque pass through the endpoints and the lumen-center, wherein the lines tangential to the region of plaque define the coverage angle of the lumen-centered arc.

Generating the lumen-centered arc can further include: storing a representation of the lumen-centered arc in memory, wherein the representation can include position data defining the position of the endpoints and the lumen-center.

The identification of the region of plaque can include a mask, wherein a region of pixels in the mask corresponds to the region of plaque in the image frame, wherein the identification can include data defining the positions of pixels in the mask corresponding to the region of plaque and expressed as polar coordinates relative to a device-center of the imaging device while the imaging device was in the lumen, and wherein the method further can include converting the positions of pixels from polar coordinates relative to the device-center, to polar coordinates relative to the lumen-center.

Converting the positions of the pixels can include: converting the positions of the pixels from polar coordinates relative to the device-center to Cartesian coordinates; and converting the positions of the pixels from Cartesian coordinates to polar coordinates relative to the lumen-center.

The method can further include: generating the identification of the region of plaque from the image frame, including processing the image frame through one or more machine learning models trained to receive the image frame and identify one or more predicted regions of plaque in the image frame.

Identifying the lumen-center can include: computing a lumen contour of the lumen; generating a spline estimate of the lumen contour; and identifying the lumen-center as an estimation of a centroid from a plurality of samples of the spline estimate.

The image frame can be a first image frame and is one of a plurality of image frames in a sequence, wherein the lumen-center is a first lumen-center, and wherein the method further can include: identifying a respective lumen-center for each of the plurality of image frames in the sequence; applying a low-pass smoothing filter to the respective lumen-center for each of the plurality of image frames, including the first image frame and the first lumen-center; and generating, using at least the lumen-center after applying the low-pass smoothing filter and endpoints of the device-centered arc, a second lumen-centered arc having a coverage angle centered on the smoothed lumen-center.

Applying the low-pass smoothing filter further can include: applying the low-pass smoothing filter over an array of lumen-centers for the plurality of images that is symmetric around the lumen-center of the image frame in the middle of the sequence, wherein the filter further can include one or more filter coefficients that are applied on a first image frame and depend at least on the relative frame index between the image frame in the middle of the sequence and the first image frame.

Identifying the respective lumen-center for each of the plurality of image frames in the sequence further can include: identifying one or more image frames depicting a respective side branch off of the lumen; and interpolating, for each of the one or more image frames, a respective lumen-center from lumen-centers of neighboring image frames in the sequence that do not depict the respective side branch.

Displaying on a display coupled to the one or more processors the image frame annotated with the lumen-centered arc.

Displaying the image frame annotated with the lumen-centered arc further can include: displaying the image frame within a field of view of a display viewport having a boundary, including displaying the lumen-centered arc along the boundary of the display viewport.

The imaging device can be an optical coherence tomography (OCT) imaging device or an intravascular ultrasound (IVUS) imaging device.

The method can further include: generating, by the one or more processors and at least partially using the coverage angle of the lumen-centered arc, one or more calcium scoring metric values corresponding to the region of plaque.

The region of plaque can be a first region of plaque of a plurality of regions of plaque in the image frame, and wherein the method further can include generating, for each of the plurality of regions of media, a respective lumen-centered arc having a respective coverage angle centered on the lumen-center.

The region of plaque can be calcium.

Aspects of the disclosure also provide for a system including: one or more processors configured to: receive an image frame and an identification of a region of plaque in the image frame, wherein the image frame is taken by an imaging device while the imaging device was in a lumen depicted in the image frame; identify a lumen-center of the lumen depicted in the image frame; and generate, using at least the lumen-center, a lumen-centered arc having a coverage angle centered on the lumen-center.

The identification of the region of plaque can include a mask, wherein a region of pixels in the mask corresponds to the region of plaque depicted in the image frame.

The one or more processors can be further configured to: identify endpoints of the region of plaque through which lines tangential to the region of plaque pass through the endpoints and the lumen-center, wherein the lines tangential to the region of plaque define the coverage angle of the lumen-centered arc.

To generate the lumen-centered arc, the one or more processors are further configured to: storing a representation of the lumen-centered arc in memory, wherein the representation can include position data defining the position of the endpoints and the lumen-center.

The identification of the region of plaque can include a mask, wherein a region of pixels in the mask corresponds to the region of plaque in the image frame, wherein the identification can include data defining the positions of pixels in the mask corresponding to the region of plaque and expressed as polar coordinates relative to a device-center of the imaging device while the imaging device was in the lumen, and wherein the one or more processors are further configured to convert the positions of pixels from polar coordinates relative to the device-center, to polar coordinates relative to the lumen-center.

Converting the positions of the pixels can further include: converting the positions of the pixels from polar coordinates relative to the device-center to Cartesian coordinates; and converting the positions of the pixels from Cartesian coordinates to polar coordinates relative to the lumen-center.

The one or more processors can be further configured to: generate the identification of the region of plaque from the image frame, including processing the image frame through one or more machine learning models trained to receive the image frame and identify one or more predicted regions of plaque in the image frame.

To identify the lumen-center, the one or more processors can be further configured to: compute a lumen contour of the lumen; generate a spline estimate of the lumen contour; and identify the lumen-center as an estimation of a centroid from a plurality of samples of the spline estimate.

The image frame can be a first image frame and is one of a plurality of image frames in a sequence, wherein the lumen-center is a first lumen-center, and wherein the one or more processors are further configured to: identify a respective lumen-center for each of the plurality of image frames in the sequence; apply a low-pass smoothing filter to the respective lumen-center for each of the plurality of image frames, including the first image frame and the first lumen-center; and generate, using at least the lumen-center after applying the low-pass smoothing filter and endpoints of the device-centered arc, a second lumen-centered arc having a coverage angle centered on the smoothed lumen-center.

To apply the low-pass smoothing filter, the one or more processors can be further configured to: apply the low-pass smoothing filter over an array of lumen-centers for the plurality of images that is symmetric around the lumen-center of the image frame in the middle of the sequence, wherein the filter further can include one or more filter coefficients that are applied on a first image frame and depend at least on the relative frame index between the image frame in the middle of the sequence and the first image frame.

To identify the respective lumen-center for each of the plurality of image frames in the sequence, the one or more processors can be further configured to: identify one or more image frames depicting a respective side branch off of the lumen; and interpolate, for each of the one or more image frames, a respective lumen-center from lumen-centers of neighboring image frames in the sequence that do not depict the respective side branch.

The method can further include: displaying the image frame annotated with the lumen-centered arc on a display coupled to the one or more processors.

To display the image frame annotated with the lumen-centered arc, the one or more processors can be further configured to: display the image frame within a field of view of a display viewport having a boundary, including displaying the lumen-centered arc along the boundary of the display viewport.

The imaging device can be an optical coherence tomography (OCT) imaging device or an intravascular ultrasound (IVUS) imaging device.

The one or more processors can be further configured to: generate, by the one or more processors and at least partially using coverage angle of the lumen-centered arc, one or more calcium scoring metric values corresponding to the region of plaque.

The region of plaque is a first region of plaque of a plurality of regions of plaque in the image frame, and wherein the one or more processors are further configured to, for each of the plurality of regions of plaque, a respective lumen-centered arc having a respective coverage angle centered on the lumen-center.

Aspects of the disclosure also provide for one or more non-transitory computer-readable storage media storing instructions that when executed by one or more processors, causes the one or more processors to perform operations including: receiving, by one or more processors, an image frame and an identification of a region of plaque in the image frame, wherein the image frame is taken by an imaging device while the imaging device was in a lumen depicted in the image frame; identifying, by the one or more processors, a lumen-center of the lumen depicted in the image frame; and generating, by the one or more processors and using at least the lumen-center, a lumen-centered arc having a coverage angle centered on the lumen-center.

The identification of the region of plaque can include a mask, wherein a region of pixels in the mask corresponds to the region of plaque in the image frame.

The operations can further include identifying endpoints of the region of plaque through which lines tangential to the region of plaque pass through the endpoints and the lumen-center, wherein the lines tangential to the region of plaque define the coverage angle of the lumen-centered arc.

Generating the lumen-centered arc further can include: storing a representation of the lumen-centered arc in memory, wherein the representation can include position data defining the position of the endpoints and the lumen-center.

The identification of the region of plaque can include a mask, wherein a region of pixels in the mask corresponds to the region of plaque in the image frame, wherein the identification can include data defining the positions of pixels in the mask corresponding to the region of plaque and expressed as polar coordinates relative to a device-center of the imaging device while the imaging device was in the lumen, and wherein the operations further include converting the positions of pixels from polar coordinates relative to the device-center, to polar coordinates relative to the lumen-center.

Converting the positions of the pixels further can include: converting the positions of the pixels from polar coordinates relative to the device-center to Cartesian coordinates; and converting the positions of the pixels from Cartesian coordinates to polar coordinates relative to the lumen-center.

The operations can further include: generating the identification of the region of plaque from the image frame, including processing the image frame through one or more machine learning models trained to receive the image frame and identify one or more predicted regions of plaque in the image frame.

Identifying the lumen-center can include: computing a lumen contour of the lumen; generating a spline estimate of the lumen contour; and identifying the lumen-center as an estimation of a centroid from a plurality of samples of the spline estimate.

The image frame is a first image frame and is one of a plurality of image frames in a sequence, wherein the lumen-center is a first lumen-center, and wherein the operations can further include: identifying a respective lumen-center for each of the plurality of image frames in the sequence; applying a low-pass smoothing filter to the respective lumen-center for each of the plurality of image frames, including the first image frame and the first lumen-center; and generating, using at least the lumen-center after applying the low-pass smoothing filter and endpoints of the device-centered arc, a second lumen-centered arc having a coverage angle centered on the smoothed lumen-center.

Applying the low-pass smoothing filter further can include: applying the low-pass smoothing filter over an array of lumen-centers for the plurality of images that is symmetric around the lumen-center of the image frame in the middle of the sequence, wherein the filter further can include one or more filter coefficients that are applied on a first image frame and depend at least on the relative frame index between the image frame in the middle of the sequence and the first image frame.

Identifying the respective lumen-center for each of the plurality of image frames in the sequence further can include: identifying one or more image frames depicting a respective side branch of the lumen; and interpolating, for each of the one or more image frames, a respective lumen-center from lumen-centers of neighboring image frames in the sequence that do not depict the respective side branch.

The operations can further include: displaying the image frame annotated with the lumen-centered arc on a display coupled to the one or more processors.

Displaying the image frame annotated with the lumen-centered arc further can include: displaying the image frame within a field of view of a display viewport having a boundary, including displaying the lumen-centered arc along the boundary of the display viewport.

The method can include: generating, based on the coverage angle and the circumference of a display port of a display device configured to display the image frame, a display angle corresponding to the coverage angle.

Generating the display angle can include generating the display angle in response to receiving an indication that a field-of-view value for the display device displaying the image frame has changed.

The one or more processors can be further configured to: generate, based on the coverage angle and the circumference of a display port of a display device configured to display the image frame, a display angle corresponding to the coverage angle.

To generate the display angle the one or more processors are further configured to: generate the display angle in response to receiving an indication that a field-of-view value for the display device displaying the image frame has changed.

The operations can further include: generating, based on the coverage angle and the circumference of a display port of a display device configured to display the image frame, a display angle corresponding to the coverage angle.

Generating the display angle can include generating the display angle in response to receiving an indication that a field-of-view value for the display device displaying the image frame has changed.

### BRIEF DESCRIPTION OF THE DRAWINGS

FIG. 1 illustrates an image frame annotated with lumen-centered calcium arcs, according to aspects of the disclosure.

FIG. 2 is a block diagram of an example image processing system, according to aspects of the disclosure.

FIG. 3 is a flow chart of an example process for calculating lumen-centered calcium arcs, according to aspects of the disclosure.

FIG. 4A illustrates device-centered calcium arcs.

FIG. 4B illustrates a calcium mask for regions of calcium shown in FIG. 4A.

FIG. 5A illustrates coverage angles from both a device-centered calcium arc and a lumen-centered calcium arc in an image frame.

FIG. 5B illustrates a calcium mask identifying a region of calcium between endpoints as shown in FIG. 5A.

FIG. 6 is a flow chart of an example process for calculating lumen-centered calcium arcs in an image frame, according to aspects of the disclosure.

FIG. 7 illustrates a calcium mask spanning the top and bottom of an image frame.

FIG. 8 is a flow chart of an example process for identifying a lumen-center for an image frame, according to aspects of the disclosure.

FIG. 9 is a flow chart of an example process for smoothing lumen-centers for a sequence of image frames, according to aspects of the disclosure.

FIG. 10 is a chart illustrating the relationship between relative frame index and filter weight.

### DETAILED DESCRIPTION

### Overview

Aspects of the disclosure provide for computing a lumen-centered calcium arc of a region of calcium in an image frame of a lumen using at least the center of the lumen.

A calcium arc is a measure of the angle in which calcium is formed in tissue around a lumen. For example, calcium in tissue around half of the lumen forms a calcium arc with a coverage angle of 180 degrees, while calcium in tissue around three-quarters of a lumen forms a calcium arc with a coverage angle of 270 degrees. Although reference is made in this specification to calcium arcs corresponding to calcium in tissue, aspects of the disclosure also include applying the techniques described to plaque and other identified regions of interest in tissue around a lumen. Other examples of regions of plaque can include fibers or lipid.

An imaging device, such as a catheter with an imaging probe can be maneuvered through a lumen and be configured to take images as a sequence of image frames. The raw image frames collected from the device are initially centered relative to the position of the probe in the lumen at the time the image was captured. In this specification, the device-center in an image frame refers to the position of the imaging probe in the lumen at the time the imaging probe captured the image frame. In examples in which the imaging device is a catheter, the device-center may also be referred to as a catheter-center, measured from the center of the catheter as shown in the image frame captured by the imaging probe. From the image frames, a system configured to process images can identify the presence of regions of plaque, such as calcium, visible in tissue around the lumen.

Some diagnostic techniques, such as calcium scoring, use at least in part information relating to calcium arcs formed by regions of calcium identified in image frames of a blood vessel and surrounding tissue. Calcium scoring refers to techniques for quantifying characteristics of calcium in plaque detected in biomedical images, such as in images of blood vessels. Calcium scoring is used to assess a patient's risk of heart attack or other cardiovascular diseases. Biomedical images used for calcium scoring can come from a device such as a catheter with an imaging probe configured to take images of a vessel while passing through the vessel.

Calcium scoring rubrics often factor in the calcium arc of a region of calcium relative to the center of the lumen in the image frame, and not the center of the device capturing the image. Calcium arcs relative to the center of the lumen are referred to in this specification as lumen-centered calcium arcs, and the center of the lumen or the lumen-center of an image frame refers to the position of the center of the lumen depicted in the image frame.

One problem is that the techniques for identifying regions of calcium often require the use of raw image data that does not identify the respective lumen-center for images in the data. Another problem even after identifying the lumen-center of an image is accurately identifying the coverage angle for the lumen-centered calcium arc.

Aspects of the disclosure provide for techniques for accurately computing lumen-centered calcium arcs from raw image data identifying regions of plaque. By computing the lumen-centered calcium arc for a region of calcium, the coverage angle for the lumen-centered calcium arc can be identified more accurately versus the coverage angle for a device-centered calcium arc on the same region of calcium. Further, computed lumen-centered calcium arcs can be displayed more consistently for a recording of image frames captured during a pullback, versus displaying device-centered arcs. Lumen-centered arcs as described herein can be displayed with according to display angles that are not distorted relative to the field-of-view in which an image frame is viewed through a display viewport. In this way, lumen-centered arcs can be displayed so as to not be subject to distortion relative to image frames viewed using different field of views, as described in more detail herein.

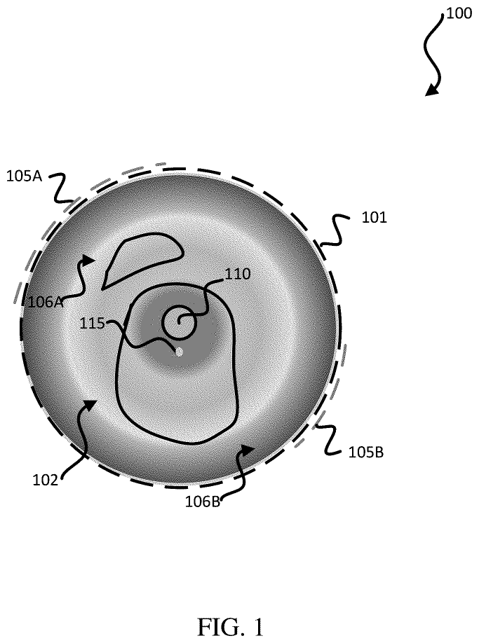

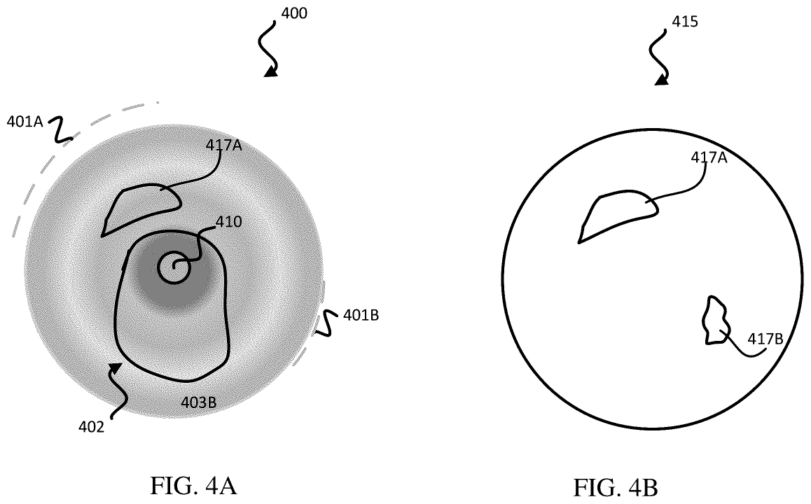

FIG. 1 illustrates an image frame 100 annotated with lumen-centered calcium arcs 105A-B, according to aspects of the disclosure. The image frame 100 also shows a lumen 102, a device-center 110 for a device (not shown) that captured the image frame 100, and a lumen-center 115. The image frame 100 can include data identifying the regions of calcium 106A-B, for example as a mask, such as the mask shown and described with reference to FIG. 4B. The image frame 100 can be displayed through a display viewport, which includes a viewport boundary 101. The viewport boundary 101 encloses a portion of the image frame 100 depicting parts of the lumen and surrounding tissue captured by the imaging device. As an example, the image frame 100 can be displayed to depict a two-dimensional cross-section of the imaged lumen. The lumen-centered calcium arcs 105A-B are drawn on the viewport boundary 101. The image frame 100 can be viewed according to different values for the field of view (FOV) of the image frame. The image frame 100 can be displayed at different FOV values, which generally correspond to how much of the lumen and surrounding tissue is visible within the viewport boundary 101 at once.

Lumen-centered calcium arcs can be annotated on image frames and can provide for a more accurate comparison between image frames taken from the same sequence by an imaging device in a lumen, relative to device-centered calcium arcs. This is at least because lumen-centered calcium arcs are based on the position of the center of the lumen, which varies less over the sequence of images versus even an expertly maneuvered imaging device through a lumen. In addition, the system is configured to compute display angles corresponding to coverage angles for lumen-centered arcs, which can be displayed without distortion from image frame-to-frame. For example, as shown in FIG. 1, the lumen-centered arcs 105A-B can be consistently drawn on the viewport boundary 101 regardless of the current FOV at which the image frame 100 is viewed. The system can detect whether the FOV of the image has been updated, and in response update a display angle to represent the coverage angle of the lumen-centered arc, which would otherwise appear distorted. The distortion can occur at least because the image frame is displayed relative to the center of the device responsible for capturing the image. Whereas the device-centered arc can be displayed with the same angle regardless of the FOV, a lumen-centered arc will not. Therefore, the system is configured to separately calculate a coverage angle for a lumen-centered arc, which can be used for calcium scoring or other downstream processing, in addition to a display angle which corresponds to the coverage angle and does not appear distorted for different FOVs.

Lumen-centered calcium arcs can also be provided as input to downstream processes for calcium scoring, and can result in more accurate scoring at least because coverage angles for the arcs are more consistently identified relative to the lumen-center.

Aspects of the disclosure also provide for techniques for identifying the lumen-center of a lumen depicted across a sequence of image frames, such as a video recording captured by an imaging device. The position of the lumen-center can be smoothed out across multiple image frames to reduce jitter and discrepancies of the lumen-center from frame-to-frame. Further, a system configured according to aspects of the disclosure can compute and provide for multiple lumen-centered calcium arcs, based on both a lumen-center and/or a smoothed lumen-center identified for an image frame.

In addition, aspects of the disclosure can be integrated more easily into existing processing pipelines, at least because only the raw image frames (which generally indicate the device-center for each frame) are required. In other words, the techniques described do not require additional pre-processing and can receive raw image data with regions of plaque identified from any of a variety of sources.

### Example Systems

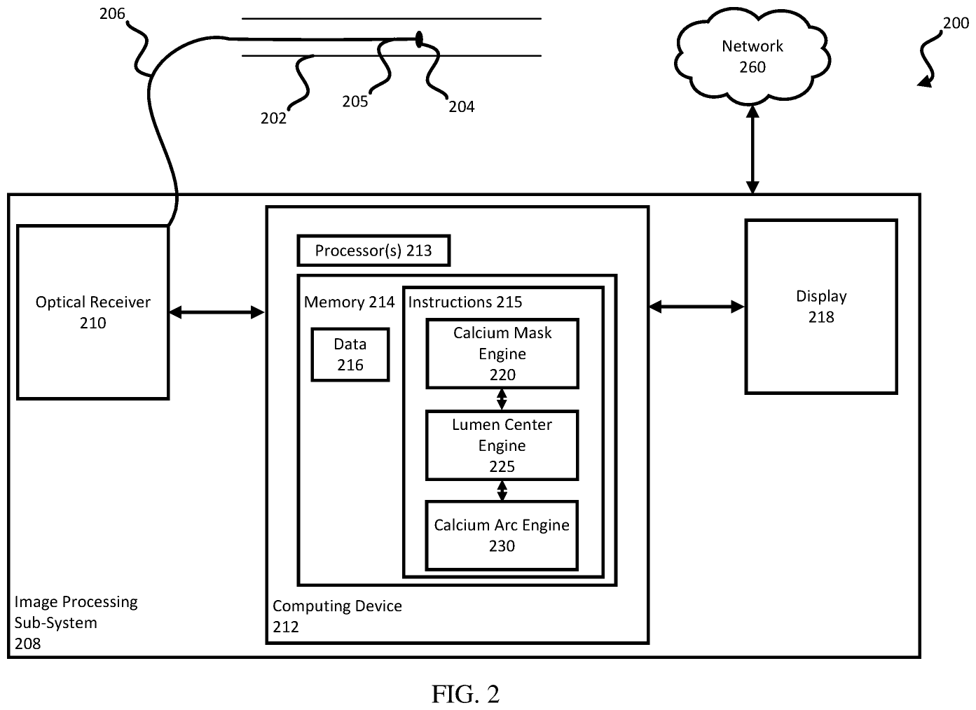

FIG. 2 is a block diagram of an example image processing system 200, according to aspects of the disclosure. The system 200 can include an imaging device 205 with an imaging probe 204 that can be used to image a lumen 202, such as a lumen of a blood vessel. The imaging device 205 can be, for example, a catheter. The imaging probe 204 may be an OCT probe and/or an IVUS catheter. While the examples provided herein refer to an OCT probe, the use of an OCT probe is not intended to be limiting. An IVUS catheter may be used in conjunction with or instead of the OCT probe. A guidewire, not shown, may be used to introduce the probe 204 into the lumen 202. The probe 204 may be introduced and pulled back along a length of a lumen while collecting data, for example as a sequence of image frames. According to some examples, the probe 204 may be held stationary during a pullback such that a plurality of scans of OCT and/or IVUS data sets may be collected. The data sets, or frames of image data, may be used to identify features, such as calcium arcs relative to a device or lumen center, as described herein.

The probe 204 may be connected to an image processing subsystem 208 through an optical fiber 206. The image processing subsystem 208 may include a light source, such as a laser, an interferometer having a sample arm and a reference arm, various optical paths, a clock generator, photodiodes, and other OCT and/or IVUS components.

The probe 204 may be connected to an optical receiver 210. According to some examples, the optical receiver 210 may be a balanced photodiode-based system. The optical receiver 210 may be configured to receive light collected by the probe 204.

The subsystem 208 may include a computing device 212. The computing device 212 may include one or more processors 213, memory 214, instructions 215, and data 216. The computing device 212 can also implement a calcium mask engine 220, a lumen center engine 225, and/or a calcium arc engine 230.

The one or more processors 213 may be any combination of a variety of different processors, such as commercially available microprocessors. Alternatively, the one or more processors may be one or more devices such as graphics processing units (GPU), field-programmable gate arrays (FPGAs), and/or application-specific integrated circuits (ASICs). Although FIG. 2 functionally illustrates the processor, memory, and other elements of device 210 as being within the same block, it will be understood by those of ordinary skill in the art that the processor, computing device, or memory may actually include multiple processors, computing devices, or memories that may or may not be stored within the same physical housing. Similarly, the memory may be a hard drive or other storage media located in a housing different from that of the computing device 212. Accordingly, references to a processor or computing device will be understood to include references to a collection of processors or computing devices or memories that may or may not operate in parallel.

Memory 214 may store information that is accessible by the processors, including instructions 215 that may be executed by the processors 213, and data 216. The memory 214 may be a type of memory operative to store information accessible by the processors 213, including a non-transitory computer-readable medium, or other medium that stores data that may be read with the aid of an electronic device, such as a hard-drive, memory card, read-only memory (“ROM”), random access memory (“RAM”), optical disks, as well as other write-capable and read-only memories. The subject matter disclosed herein may include different combinations of the foregoing, whereby different portions of the instructions 215 and data 216 are stored on different types of media.

Memory 214 may be retrieved, stored, or modified by processor(s) 213 in accordance with the instructions 215. For instance, although the present disclosure is not limited by a particular data structure, the data 216 may be stored in computer registers, in a relational database as a table having a plurality of different fields and records, XML documents, or flat files. The data 216 may also be formatted in a computer-readable format such as, but not limited to, binary values, ASCII or Unicode. By further way of example only, the data 216 may be stored as bitmaps that include pixels that are stored in compressed or uncompressed, or various image formats (e.g., JPEG), vector-based formats (e.g., Scalable Vector Graphics (SVG)) or computer instructions for drawing graphics. Moreover, the data 216 may include information sufficient to identify the relevant information, such as numbers, descriptive text, proprietary codes, pointers, references to data stored in other memories (including other network locations) or information that is used by a function to calculate the relevant data.

The instructions 215 can be any set of instructions to be executed directly, such as machine code, or indirectly, such as scripts, by the processor(s) 213. In that regard, the terms “instructions,” “application,” “steps,” and “programs” can be used interchangeably herein. The instructions can be stored in object code format for direct processing by the processor, or in any other computing device language including scripts or collections of independent source code modules that are interpreted on demand or compiled in advance. Functions, methods and routines of the instructions are explained in more detail below, including description of the calcium mask engine 220, the lumen center engine 225, and the calcium arc engine 230.

The computing device 212 can receive one or more image frames collected by the imaging probe 204 while maneuvered through the lumen 202. The computing device 212 can be configured to process incoming raw image frames through the calcium mask engine 220. The calcium mask engine 220 is configured to receive image frames as input, and identify one or more regions (or “blobs”) of plaque in the image. To do so, the calcium mask engine 220 can implement any of a variety of image processing techniques, including one or more machine learning models trained to perform image segmentation on input image data. While the subsystem 208 is described as implementing the engine 220 for identifying calcium, it is understood that the subsystem 208 can be configured for any image segmentation task, such as to generally identify one or more regions of plaque in an input image frame.

Although the subsystem 208 is described as processing the raw image frames received from the imaging probe 204 through the optical receiver 210, in some implementations the subsystem 208 receives image frames with one or more regions of plaque identified. In other words, instead of processing the image frames to identify the regions of plaque, the subsystem 208 can be configured to receive processed images from another device, such as an image processing device, storage device, etc.

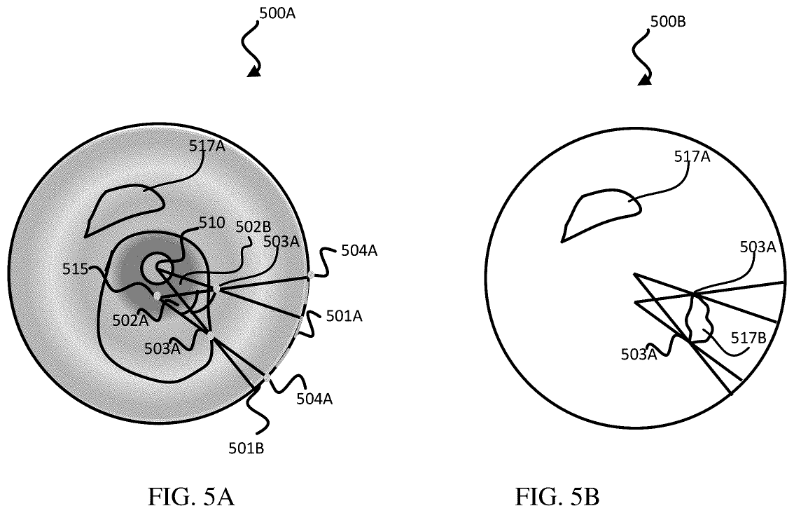

The identification of a region of plaque of an image can be represented in a number of ways. For example, the region of plaque may be annotated in the image frame with some visual indicator, such as an outline, perimeter, or shaded portion of pixels. The annotations may be included in an image overlay, directly added to the image, or included in any other way. In addition or alternatively, the image frame can be accompanied by a calcium mask. The calcium mask can correspond to pixels in the image frame that are predicted to correspond to a region of calcium. FIGS. 4B and 5B, described below, illustrate examples of calcium masks over predicted regions of calcium in respective image frames.

In some examples, the region of plaque may not be annotated in the image frame, and engine 225 instead is configured to identify a perimeter for the region, instead. In examples in which the region of plaque is not annotated, the engine 225 can identify a perimeter of a region of plaque. In some examples, the engine 225 stores data indicating the position of pixels representing an identified region of plaque or perimeter for an identified region. The data can be, for example, a mask, a list of pixel positions, etc. In some examples, the engine 225 can generate a visual annotation of the region after identification. In examples in which the engine 225 does not receive a mask or other data indicating the location of various different regions of plaque, the engine 225 can instead perform its own identification and generate data as described herein for identifying the regions of plaque.

In one example, the engine 225 can process scanlines or rows of pixels in a received image, and identify the intensity of backscatter from each pixel, and/or edges based on attenuation computed from the pixel intensity gradient along the scanline. The engine 225 can, from the measured intensity and/or attenuation, identify to which type of tissue, lesion, or plaque a pixel corresponds to. For example, higher intensities, e.g., brighter reflections of backscatter, and lower attenuations, e.g., lower changes in intensity from pixel to pixel, may correspond to fibrous tissues. Lower intensity, lower attenuation, and sharper edges between pixels measured with different intensities/attenuations can correspond to a region of calcium. Lower intensity, higher attenuation, and diffused edges between pixels measured with different intensities/attenuations can correspond to regions of lipid, and this motivates the determination of lipid offset using edges as described above. The engine 225 can be configured with various predetermined thresholds or adaptive gradient detection algorithms for intensity and/or attenuation, for use in comparing between lower intensity and higher intensity, lower attenuation and higher attenuation, etc., while processing an image. This can enable the software to identify not only scanlines which contain various morphologies (such as lipid), but the offset at which the morphology is to be found. The scanline/offset information can be used to generate a lumen centered morphology arc similar to the calcium arc in cases such as lipid detection where scanline based classification works but no mask exists to perform the analysis.

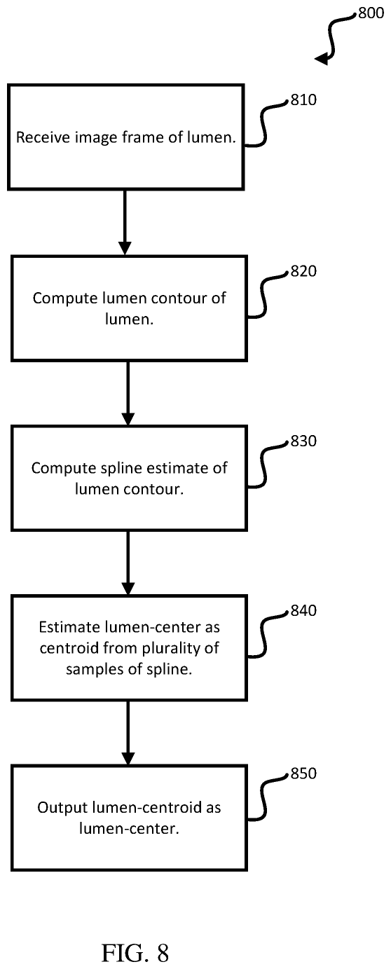

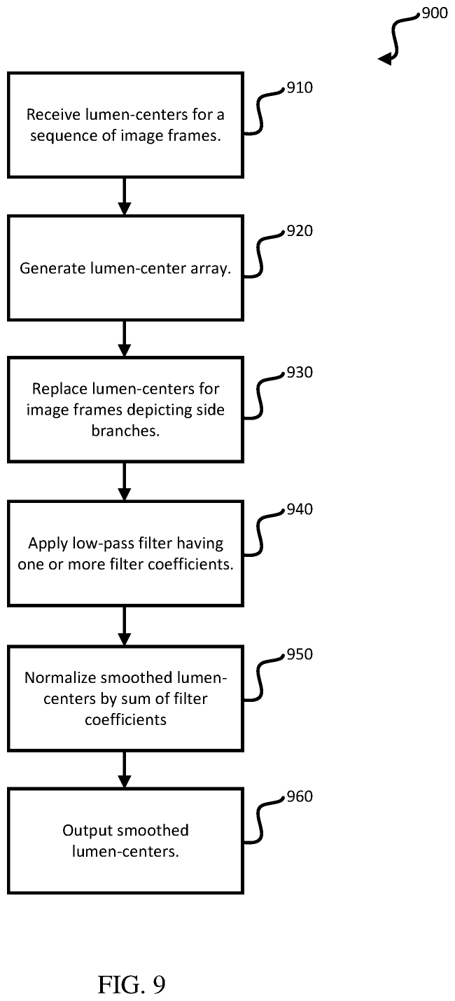

The lumen center engine 225 can be configured to receive image frames and compute a respective lumen-center for each image frame, as well as smooth lumen-centers across a sequence of image frames. As described in more detail herein with reference to FIGS. 8 and 9, aspects of the disclosure provide for techniques for identifying a lumen-center in an individual image frame, and applying a smoothing filter over the positions of lumen-centers across a sequence of image frames.

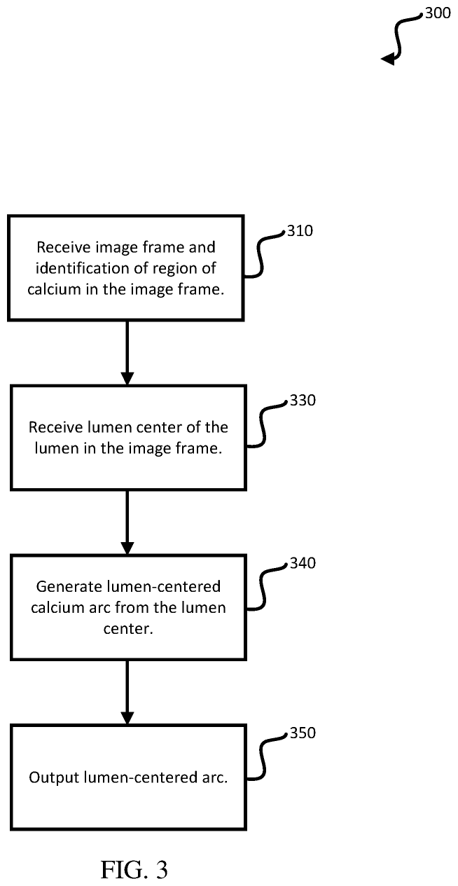

The calcium arc engine 230 can be configured to compute lumen-centered calcium arcs, for example and as described in more detail with reference to FIGS. 3-6. The calcium arc engine 230 can receive, as input, image frames captured from the imaging probe 204 through the optical receiver 210. The calcium arc engine 230 can also receive as input data identifying one or more regions of calcium in the received image frames, which for example can be generated by the calcium mask engine 220. The calcium arc engine 230 can also receive, for example from the lumen center engine 225, data corresponding to the lumen-centers and/or smoothed lumen-centers of received image frames.

The calcium arc engine 230 can generate, as output, data corresponding to a generated lumen-centered calcium arc, for each image frame and for each region of calcium in the image frame. The calcium arc engine 230 can provide the lumen-centered calcium arcs as visual annotations to received image frames, for example as shown in FIG. 1. In some implementations, the calcium arc engine 230 can adjust the lumen-centered calcium arc for display on a monitor, based on the field-of-view of a display viewport through which images of a lumen are displayed.

Alternatively or in addition, the calcium arc engine 230 can provide data corresponding to the calculated lumen-centered calcium arcs to a downstream process configured to receive and process the lumen-centered calcium arcs. The downstream process can be implemented as one or more computer programs on the device 212, or on another device altogether. The process can, for example, receive the data corresponding to the calculated lumen-centered calcium arcs and the image frames, and use the data as part of a process for calcium scoring the regions of calcium identified in the image frames.

The image processing subsystem 208 may include a display 218 for outputting content to a user. As shown, the display 218 is separate from the computing device 212 however, according to some examples, display 218 may be part of the computing device 212. The display 218 may output image data relating to one or more features detected in the lumen. For example, the output may include, without limitation, the image frames annotated with lumen-centered calcium arcs, lumen-centers, and/or smoothed lumen-centers, for each region of calcium identified in the image frames. The display 218 can display output in some examples through a display viewport, such as a circular display viewport. The display 218 can show the image frames in sequence relative to a field-of-view value.

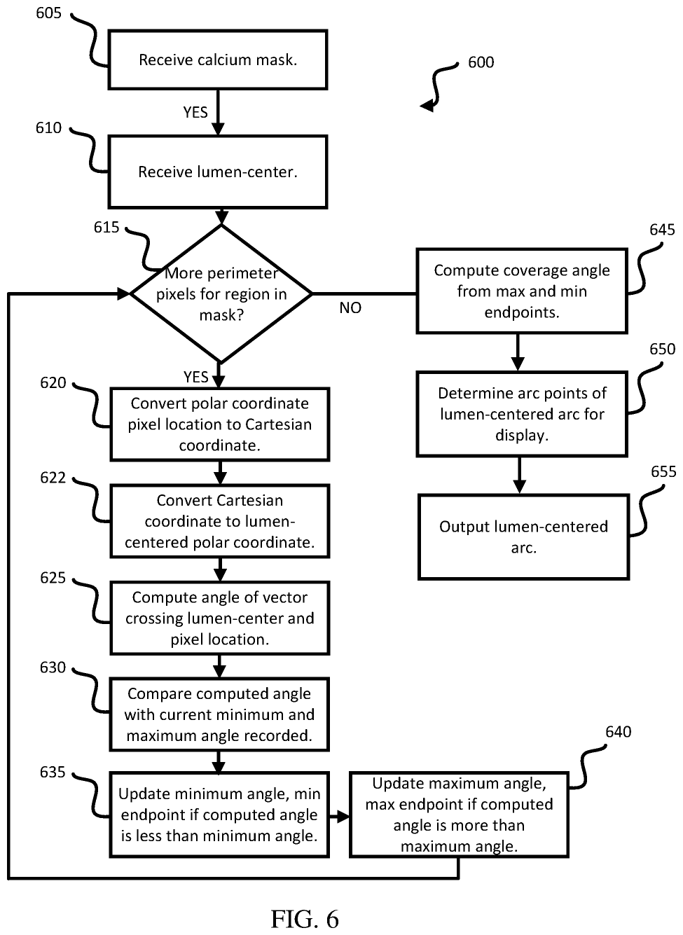

As described herein with reference to FIG. 6, the system 200 can be configured to compute display angles which correspond to coverage angles of a lumen-centered arc, accounting for the field-of-view value in which the display 218 is currently set to. In response to input to adjust the FOV, the system 200 can be configured to subsequently recalculate the display angle of a corresponding coverage angle of a region of calcium currently on display. The display 218 can be configured in some implementations to output both the display angle and the coverage angle corresponding to the display angle. In some examples, the coverage angle value is indicated but an arc corresponding to the display angle is shown on the display 218.

The display 218 can show one or more image frames, for example as two-dimensional cross-sections of the lumen 202 and surrounding tissue being imaged. The display 218 can also include one or more other views to show different perspectives of the imaged lumen or another region of interest in the body of a patient. As an example, the display 218 can include a longitudinal view of the length of the lumen 202 from a start point to an end point. In some examples, the display 218 can highlight certain portions of the lumen 202 along the longitudinal view, and at least partially occlude other portions that are not currently selected for view. In some examples the display 218 is configured to receive input to scrub through different portions of the lumen 202 as shown in the longitudinal view.

The output can be displayed in real-time, for example during a procedure in which the imaging probe 204 is maneuvered through the lumen 202. Other data that can be output, for example in combination with the above-mentioned lumen-centered arcs, can include device-centered arcs, cross-sectional scan data, longitudinal scans, diameter graphs, lumen borders, plaque sizes, plaque circumference, visual indicia of plaque location, visual indicia of risk posed to stent expansion, flow rate, etc. The display 218 may identify features with text, arrows, color coding, highlighting, contour lines, or other suitable human or machine readable indicia.

According to some examples the display 218 may be a graphic user interface (“GUI”). One or more steps may be performed automatically or without user input to navigate images, input information, select and/or interact with an input, etc. The display 218 alone or in combination with computing device 212 may allow for toggling between one or more viewing modes in response to user inputs. For example, a user may be able to toggle between different side branches on the display 218, such as by selecting a particular side branch and/or by selecting a view associated with the particular side branch.

In some examples, the display 218, alone or in combination with computing device 212, may include a menu. The menu may allow a user to show or hide various features. There may be more than one menu. For example, there may be a menu for selecting lumen features to display. Additionally or alternatively, there may be a menu for selecting the virtual camera angle of the display. In some examples the display 217 can be configured to receive input. For example, the display 217 can include a touchscreen configured to receive touch input for interacting with a menu or other interactable element displayed on the display 217.

The computing device 212 can be capable of direct and indirect communication with other devices over a network 260. The computing device 212 can set up listening sockets that may accept an initiating connection for sending and receiving information. The network 260 itself can include various configurations and protocols including the Internet, World Wide Web, intranets, virtual private networks, wide area networks, local networks, and private networks using communication protocols proprietary to one or more companies. The network can support a variety of short- and long-range connections. The short- and long-range connections may be made over different bandwidths, such as 2.402 GHz to 2.480 GHz (commonly associated with the Bluetooth® standard), 2.4 GHz and 5 GHz (commonly associated with the Wi-Fi® communication protocol); or with a variety of communication standards, such as the LTE® standard for wireless broadband communication. The network 260, in addition or alternatively, can also support wired connections between the device 212 and one or more other devices (not shown), including over various types of Ethernet connection. For example, the device 212 can communicate image frames annotated with lumen-centered arcs to one or more devices configured to process the annotated frames. In addition or alternatively, the device 212 can communicate among one or more other devices implementing at least a portion of the system 200. As another example, the device 212 can receive image frames over the network 260 and from an optical receiver connected to a device separate from the device 212.

### Example Methods

FIG. 3 is a flow chart of an example process 300 for calculating lumen-centered calcium arcs, according to aspects of the disclosure. A system having one or more processors and appropriately programmed in accordance with aspects of the disclosure can perform the process 300. For example, an image processing system, such as the image processing system 200 of FIG. 2 can perform the process 300.

The system receives an image frame and identification of a region of calcium in the image frame, according to block 310. As described herein with reference to FIG. 2, a calcium mask engine for the system can be configured to process raw image frames from the imaging probe and generate calcium masks for each frame. In other examples, the system can receive the image frames and corresponding masks or other data identifying regions of calcium from another device altogether. Although described with reference to a single image frame, the system can be configured to receive multiple image frames, for example as a stream of images in real-time during an imaging procedure, or as a batch of data. In either case, the system can be configured to repeat the process 300 for each image frame, for example sequentially or in parallel.

The system receives the lumen-center of the lumen in the image frame, according to block 330. The system can be configured to calculate a lumen-center and/or smoothed lumen-center for a lumen, for example using the lumen center engine configured to perform the processes described herein with reference to FIGS. 8 and 9. The lumen-center can be represented, for example, in polar coordinates. Polar coordinates can be represented by two parameters relative to a reference point, the radius and angle, which can be initially relative to the device-center.

The system generates a lumen-centered calcium arc from the lumen-center of the lumen in the image frame, according to block 340. The system can convert the lumen-center from polar coordinates relative to the device-center, to Cartesian coordinates. For example, the system can compute:

x=radius*cos(θ) [1]

y=radius*sin(θ) [2]

to compute Cartesian coordinates for a point expressed in polar coordinates.

As part of generating the lumen-centered calcium arc, the system identifies endpoints of the region of calcium, as described herein with reference to FIGS. 5A-B, 6. The endpoints and the lumen-center define lines that are tangential to the region of calcium. The system can compute Cartesian coordinates for the endpoints of a device-centered calcium arc, and the lumen-center. The endpoints of the lumen-centered calcium arc, along with the lumen-center, define the correct coverage angle for the arc relative to a region of calcium.

The system can compute the coverage angle from the start and end angle of the lumen-centered calcium arc for the region of calcium. For example, the system can compute the coverage angle for the lumen-centered calcium are as follows:

$$θ = arctan ( \frac{{y}_{1} - {y}_{2}}{{x}_{1} - {x}_{2}} ) \qquad [ 3 ]$$

where (x₁, y₁) are the coordinates for one endpoint, and (x₂, y₂) are the coordinates for the other endpoint. Although examples are provided using polar coordinates converted to Cartesian coordinates, in general any system of coordinates for identifying positions on an image frame can be used to express the positions of the endpoints, the lumen-center, the device-center, regions of plaque or calcium in the image frame, etc. As another example, the lumen-center and the endpoints can be expressed in Cartesian coordinates initially, instead of as polar coordinates. The image frame when displayed can include an overlay, such as a grid, corresponding to a coordinate system used to express different positions on the image frame. The lumen-centered arc can be represented in data as endpoints represented in polar coordinates, and centered on the position of device-center in the image frame.

In implementations in which both a lumen-center and a smoothed lumen-center are computed for an image frame, the system can compute a respective lumen-centered calcium arc according to block 340 for both centers.

The system outputs the lumen-centered arc (or arcs, for multiple regions of calcium in the image frame), according to block 350. For example, and as described with reference to FIG. 2, the system can output the lumen-centered arc on a display as an annotation over the image frame. The system can draw the lumen-centered arcs over the image frame and along the viewport boundary of a display viewport used to display the image frame. For example, the system can project the tangential lines defined by the endpoints of the region of calcium and the lumen-center through the boundary. The system can annotate the portion of the boundary between the lines projected through the boundary as the lumen-centered calcium arc.

In other examples, the system can output data defining the lumen-centered calcium arc for downstream processing, for example for calcium scoring. The calcium scoring can be performed automatically, by the system or another system, or the calcium scoring can be done by hand, as examples. The system can output both lumen-centered calcium arcs for one or both of a lumen-center and a smoothed lumen-center, in implementations in which both are computed.

FIG. 4A illustrates device-centered calcium arcs 401A-B. Device-center 410 is shown in the upper part of a lumen 402 captured in an image frame 400.

FIG. 4B illustrates a calcium mask 415 for regions of calcium shown in FIG. 4A. The calcium mask 415 includes calcium identifiers 417A and 417B, each corresponding to a respective identified region of calcium identified in the image frame 400. In this example, the calcium identifiers 417A-B are regions of pixels of a particular color value to distinguish the regions of calcium from other regions of the image frame 400, such as regions depicting the lumen 402 or media surrounding the lumen 402. The calcium mask 415 is an example of an identification of regions of calcium in the image frame 400, and can be received by the system, for example from a calcium mask engine implemented as part of the system, or from another source.

The calcium identifiers can be expressed in any of a variety of different ways. In some examples, the calcium identifiers can be expressed as a cross-thatched or patterned region of pixels, or as pixels corresponding to the outline of the detected region of calcium. In other examples, the calcium identifiers are represented as a matrix of values, each value corresponding to a pixel in the image frame. Each value can be binary, for example with a value of zero indicating that the corresponding pixel is not of a portion of the region of calcium, and a value of one indicating that the corresponding pixel is of a portion of the region of calcium.

FIG. 5A illustrates coverage angles 502A-B for both a device-centered calcium arc 501A and a lumen-centered calcium arc 501B in an image frame 500A. The image frame 500A includes a lumen-center 515 and a device-center 510. Endpoints 503A are the endpoints of a region of calcium, for example computed as described herein with reference to FIGS. 3 and 6. Arc points 504A are points at which lines tangential to the region of calcium pass through the viewport boundary (not shown). Using the arc points 504A, the system can annotate for display the lumen-centered calcium arc 501B along the viewport boundary. Also shown is the device-centered calcium arc 501A. Unlike the lumen-centered calcium arc 501B, the device-centered calcium arc 501A can shift along the viewport boundary for different FOV values of the image frame when displayed.

The system can leverage a known device-center to compute endpoints of a region of calcium and a lumen-center to arrive at a lumen-centered calcium arc that is more accurate than a device-centered calcium arc. In this way, the system can process image data from a variety of different sources that may include models for predicting features of the image data, such as a calcium mask. In other words, the system is more compatible with processing on raw image data, at least because it is configured to calculate the lumen-center and lumen-centered calcium arcs as post-processing steps.

FIG. 5B illustrates a calcium mask 500B identifying a region of calcium between endpoints 503A. FIG. 5B illustrates the relationship between the endpoints 503A as being spaced along the maximum width of the region of calcium identified by calcium identifier 517B. Calcium mask 500B also shows a region of calcium identified by calcium identifier 517A. FIG. 6, herein, illustrates a process for computing the endpoints 503A.

FIG. 6 is a flow chart of an example process 600 for identifying lumen-centered calcium arcs in an image frame, according to aspects of the disclosure. A system, such as the system 200 of FIG. 2, can perform the process 600.

The system receives a calcium mask, according to block 605. For ease of description, the calcium mask is assumed to identify a single region of calcium, although it is understood that the system can perform the process 600 for each region identified in a calcium mask.

The system receives the position of a lumen-center for an image frame corresponding to the calcium mask, according to block 610. The lumen-center can be computed, for example, as described herein with reference to FIG. 8. The lumen-center can also be a smoothed lumen-center, generated for example according to the process 900 in FIG. 9.

The system determines whether there are more perimeter pixels identifying the region of calcium in the mask, according to block 615. Perimeter pixels can be pixels along the perimeter of an identified region, such as pixels of the perimeter of the identifier 417B in FIG. 4B, as an example. The system can iterate over each of these perimeter pixels.

If the system determines there are more perimeter pixels for processing (“YES”), the system converts a polar coordinate pixel location to Cartesian coordinates, according to block 620. The polar coordinate pixel location is relative to the device-center in the image frame, that is, with the device-center as the center from which the radius and angle of the polar coordinates are expressed. The system can receive, as part of data defining the calcium mask, coordinate data corresponding to the locations of pixels collectively identifying a region of calcium. The coordinate data can be relative to the device-center initially, at least because the device-center is available in raw image frames that are processed to generate the corresponding calcium mask.

The system converts the Cartesian coordinate converted according to block 620, to lumen-centered polar coordinates, according to block 622. As described herein, any coordinate system can be used in place of polar coordinates. In some implementations, the system is configured to perform any necessary conversion from converting from a starting coordinate system, such as a device-centered polar coordinate system, to an ending coordinate system, such as a lumen-centered polar coordinate system, The system can convert the coordinates across intermediate coordinate systems, such as Cartesian coordinates from the device-centered polar coordinates of a perimeter pixel.

The system computes the angle of a vector crossing both the lumen-center and pixel location, according to block 625. The angle computed can be relative to a common axis, and the system can also check to account for wrapping about the common axis, and accordingly adjusting the angle measurement by adding or subtracting 360 degrees, as needed.

The system compares the computed angle with a current minimum angle and maximum angle, according to block 630. Initially, the current minimum and maximum angle can be set to the value of the angle formed by a vector though the first computed pixel location, according to block 625. The system updates the minimum angle and a minimum endpoint if the computed angle is less than the minimum angle, according to block 635. The minimum endpoint tracks the pixel location of the pixel corresponding to the current minimum angle. Similarly, the system updates the maximum angle and a maximum endpoint if the computed angle is greater than the current maximum angle, according to block 640.

Returning to block 615, if the system determines that there are no more perimeter pixels to process (“NO”), then the system computes the coverage of the lumen-centered calcium arc using the current maximum and minimum endpoints, according to block 645. For example, the system can compute the coverage angle as described herein with reference to FIG. 3. The endpoints are tangential to the identified region of calcium, for example as illustrated in FIG. 5B with the endpoints 503A.

The system determines arc points of the lumen-centered calcium arc for display, according to block 650. The arc points are used to identify the display angle corresponding to the coverage angle calculated, for example according to block 645. For example, the arc points can be the arc points 504A that represent the intersection between lines passing through the lumen-center, and a boundary of a display viewport. Computing the arc points can be useful for better displaying the lumen-centered calcium arc on a display having a viewport set to a particular field-of-view, as described herein.

The arc points can be computed by identifying the point at which lines through the lumen-center and the endpoints intersect the circular display viewport, when the image frame is displayed. For example, the system can be configured to represent the circumference of a display viewport as a circle in Cartesian coordinates, for example relative to the device-center shown in an image frame. The system can be configured to compute where two lines that intersect the lumen-center also intersect the circumference of the display port itself. As an example, the system can represent the lines and circumference of the viewport as standard equations in Cartesian space (e.g., y=mx+b for the lines; x²+y²=r² for the circle formed by the circumference of the display viewport), and solve for the intersections of the line and circle equations. Each line will intersect the circumference at exactly two points.

The system can identify the set of points closest to a region of calcium corresponding to the coverage angle calculated for example according to block 645, and select those points as the arc points for the display angle. The other two points may be discarded, or in some examples the system uses the coverage angle to identify which of the two sets of points are the arc-points for the display angle. The system can compute the display angle formed by the lumen-center, and the two arc-points. The system can further be configured to display the arc formed by the display angle. The system can be configured to repeat calculating the display angle as described with respect to block 650, for example in response to input received to a display device displaying the image frame. In some examples, if the system receives input indicating that the FOV value has changed, such as in response to user input, the system can automatically recalculate the display angle as described herein in response, and display the updated arc according to the display angle.

The system outputs the lumen-centered calcium arc, according to block 655. The system can display the lumen-centered calcium arc as part of data displayed through a display viewport of a device. The lumen-centered calcium arc can be drawn using the arc points and along the radius of the display viewport. Using the arc points and their corresponding coverage angle to the lumen-center instead of the endpoints can help to account for variations in the field-of-view for the display viewport. The coverage angle used to display the lumen-centered calcium arc on the display based on the display angle defined by the arc points and can be different from the coverage angle based on the endpoints sent downstream for further processing, for example as part of calcium scoring.

In some examples, the system checks for regions of calcium along the top and bottom of an image frame, and combines representation of the regions as a single region with a corresponding lumen-centered arc.

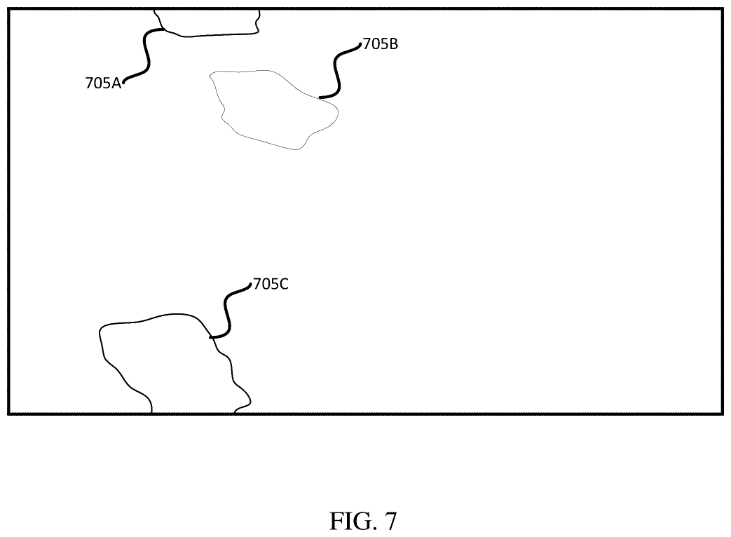

FIG. 7 illustrates a calcium mask 700 spanning the top and bottom of an image frame. Calcium identifiers 705A-C are also shown. As described herein with reference to FIG. 6, the system combines arcs formed from identifiers spanning the top and bottom of an image frame, which in the calcium mask 700 corresponds to the identifiers 705A and 705C.

FIG. 8 is a flow chart of an example process 800 for identifying a lumen-center for an image frame, according to aspects of the disclosure. A system, for example the system 200 of FIG. 2, can perform the process 800.

As with the process 300, the process 800 is described as performed on a single image frame. It is understood that the system can be configured to receive multiple image frames and repeat the process 800 for each frame to generate a respective lumen-center.

The system receives an image frame of a lumen, according to block 810.

The system computes a lumen contour of the lumen in the image frame, according to block 820. The lumen contour can be an outline defining the perimeter of the lumen. The system can approximate the lumen contour as a circle, oval, or polygon, as examples.

The system computes a spline estimate of the lumen contour, according to block 830. The spline estimate is a function of one or more polynomials that approximates the shape of the lumen contour. The system can apply any of a variety of different techniques for estimating the spline of the lumen contour. For example, the spline can be interpolated or approximated from multiple points of the lumen contour. In some examples, the system computes the lumen contour and spline estimate together. The system can generate the spline estimate expressed as polar coordinates relative to the device-center in the image frame, as the device-center is available as a reference point in every image frame.

The system estimates the lumen-center as a centroid from a plurality of samples of the spline estimate, according to block 840. Each sample can be a point on the spline estimate. Cartesian coordinates (xᵢ, yᵢ) can be the coordinates of the i-th sample. If the samples are not already expressed as Cartesian coordinates, the system can first convert each sample as Cartesian coordinates. The mean of each dimension is calculated for each dimension and over the number of samples, as shown in equations 4-5:

$$\overline{x} = \frac{1}{N} {\sum }_{i = 0}^{N - 1} {x}_{i} \qquad [ 4 ]$$

$$\overline{y} = \frac{1}{N} {\sum }_{i = 0}^{N - 1} {y}_{i} \qquad [ 5 ]$$

Equations [4] and [5] show the calculation of the means along the x-dimension (x̅) and the y-dimension (y̅), where N is the number of samples. The system can operate on different numbers of samples, from implementation-to-implementation.

Next, second and third order moments are computed, as shown below, with equations 6-12:

$${S}_{x , x} = {\sum }_{i = 0}^{N - 1} {( {x}_{i} - \overline{x} )}^{2} \qquad [ 6 ]$$

$${S}_{y , y} = {\sum }_{i = 0}^{N - 1} {( {y}_{i} - \overline{y} )}^{2} \qquad [ 7 ]$$

$${S}_{x , y} = {\sum }_{i = 0}^{N - 1} ( {x}_{i} - \overline{x} ) ( {y}_{i} - \overline{y} ) \qquad [ 8 ]$$

$${S}_{x , x , x} = {\sum }_{i = 0}^{N - 1} {( {x}_{i} - \overline{x} )}^{3} \qquad [ 9 ]$$

$${S}_{y , y , y} = {\sum }_{i = 0}^{N - 1} {( {y}_{i} - \overline{y} )}^{3} \qquad [ 10 ]$$

$${S}_{x , x , y} = {\sum }_{i = 0}^{N - 1} {( {x}_{i} - \overline{x} )}^{2} ( {y}_{i} - \overline{y} ) \qquad [ 11 ]$$

$${S}_{y , y , x} = {\sum }_{i = 0}^{N - 1} ( {x}_{i} - \overline{x} ) {( {y}_{i} - \overline{y} )}^{2} \qquad [ 12 ]$$

The system can then compute coordinates for an estimated lumen-centroid calculated from the samples and the computed second and third order moments from equations 6-12. Following equations 13-16, below:

$${B}_{x} = \frac{1}{2} ( {S}_{x , x , x} + {S}_{y , y , x} ) \qquad [ 13 ]$$

$${B}_{y} = \frac{1}{2} ( {S}_{y , y , y} + {S}_{x , x , y} ) \qquad [ 14 ]$$

$${μ}_{x} = \overline{x} + \frac{{B}_{x} {S}_{y , y} - {B}_{y} {S}_{x , y}}{{S}_{x , x} {S}_{y , y} - {S}_{x , y}^{2}} \qquad [ 15 ]$$

$${μ}_{y} = \overline{y} + \frac{{B}_{y} {S}_{x , x} - {B}_{x} {S}_{x , y}}{{S}_{x , x} {S}_{y , y} - {S}_{x , y}^{2}} \qquad [ 16 ]$$

where the coordinates for the estimated lumen-centroid are (μₓ,μ_{y}) as shown in equations 15-16.

The system outputs the centroid as the lumen-center, according to block 850. The lumen-center can be used to generate a lumen-centered calcium arc for a region of calcium in the image frame, as described herein with reference to FIG. 3. The lumen-centers of a sequence of frames can be further smoothed, as described herein with reference to FIG. 9.

FIG. 9 is a flow chart of an example process 900 for smoothing lumen-centers for a sequence of image frames, according to aspects of the disclosure. A system, such as the system 200 of FIG. 2, can perform the process 900.

The system receives coordinates defining lumen-centers for a sequence of image frames, according to block 910. As described herein with reference to FIG. 8, the system can compute lumen-centers from centroids for each image frame of a sequence of frames.

The system generates a lumen-center array, according to block 920. The lumen-center array can be symmetric around the lumen-center for the middle image frame. For example, if the sequence of images includes five frames, then the lumen-center for frame 3 is the value in the middle of the lumen-center array.

The system detects and filters out image frames depicting side branches to the imaged lumen, and replace the respective lumen-center for each filtered image frame with a respective substitute lumen-center, according to block 930. In doing so, the system can mitigate interference in smoothing the lumen-centers that can be caused by additional information from side branches depicted in the image frames. As an example, the substitute lumen-center for an image frame depicting a side branch can be linearly interpolated using positions of lumen-centers in neighboring image frames in the sequence.

The system can pad the lumen-center array with zeros. Padding the lumen-center array is done prior to applying the low-pass filter of one or more filter coefficients, according to block 940. The value of the filter coefficients can be symmetric relative to the middle frame lumen-center and vary in proportion to the relative frame index to the middle image frame. A low-pass filter modifies filtered values according to filter coefficients, to generally smoothen a distribution of values.

For example, a filter for a lumen-center array for a nine image frame sequence can have the following coefficients, according to TABLE 1:

TABLE 1

| Relative Frame Index | Filter Coefficient |
|---|---|
| −4 | −60 |
| −3 | 1686 |
| −2 | 4698 |
| −1 | 6506 |
| 0 | 7108 |
| +1 | 6506 |
| +2 | 4698 |
| +3 | 1686 |
| +4 | −60 |

For example, a lumen-center in the array two places away relative to the lumen-center at index 0 is filtered with a filter coefficient (or weight) of 4698. As another example, a lumen-center in the array four places away relative to the middle image frame is filtered with a filter coefficient of −60.

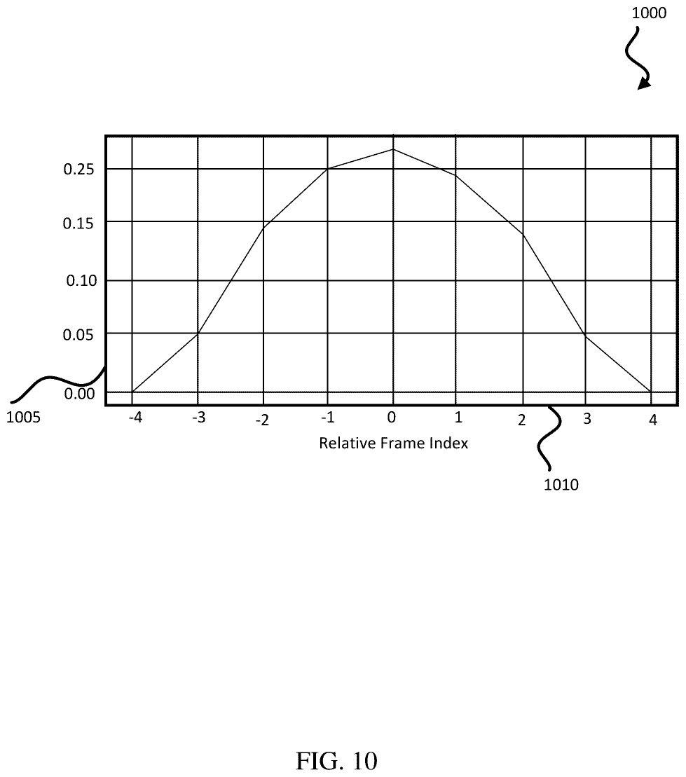

FIG. 10 is a chart 1000 illustrating the relationship between relative frame index 1005 and filter weight 1010. The y-axis represents the filter weight 1010, and the x-axis represents the relative frame index 1005 relative to the lumen-center for the middle image frame. Note the chart 1000 is symmetric relative to the middle image frame. Also note that the filter coefficient value tapers off for image frames farther away from the middle image frame, reflecting the gradually diminishing of the lumen-center smoothing from the middle image frame.

Returning to FIG. 9, as part of applying the low-pass filter according to block 940, the system can apply the filter separately for each dimension of the lumen-center, for example, along each Cartesian coordinate-dimension for each lumen-center. In some implementations, the system applies the filter twice for each dimension.

The system normalizes the smoothed lumen-centers by sum of filter coefficients, according to block 950. Normalization helps to create a zero DC gain arising from smoothing.

The system outputs the smoothed lumen-centers, according to block 960. As described herein with reference to FIGS. 2 and 3, the system can be configured to generate lumen-centered calcium arcs for both lumen-centers and smoothed lumen-centers of input image frames.

Aspects of this disclosure can be implemented in digital circuits, computer-readable storage media, as one or more computer programs, or a combination of one or more of the foregoing. The computer-readable storage media can be non-transitory, e.g., as one or more instructions executable by a cloud computing platform and stored on a tangible storage device.

In this specification the phrase “configured to” is used in different contexts related to computer systems, hardware, or part of a computer program, engine, or module. When a system is said to be configured to perform one or more operations, this means that the system has appropriate software, firmware, and/or hardware installed on the system that, when in operation, causes the system to perform the one or more operations. When some hardware is said to be configured to perform one or more operations, this means that the hardware includes one or more circuits that, when in operation, receive input and generate output according to the input and corresponding to the one or more operations. When a computer program, engine, or module is said to be configured to perform one or more operations, this means that the computer program includes one or more program instructions, that when executed by one or more computers, causes the one or more computers to perform the one or more operations.

While operations shown in the drawings and recited in the claims are shown in a particular order, it is understood that the operations can be performed in different orders than shown, and that some operations can be omitted, performed more than once, and/or be performed in parallel with other operations. Further, the separation of different system components configured for performing different operations should not be understood as requiring the components to be separated. The components, modules, programs, and engines described can be integrated together as a single system, or be part of multiple systems.

Unless otherwise stated, the foregoing alternative examples are not mutually exclusive, but may be implemented in various combinations to achieve unique advantages. As these and other variations and combinations of the features discussed above can be utilized without departing from the subject matter defined by the claims, the foregoing description of the embodiments should be taken by way of illustration rather than by way of limitation of the subject matter defined by the claims. In addition, the provision of the examples described herein, as well as clauses phrased as “such as,” “including” and the like, should not be interpreted as limiting the subject matter of the claims to the specific examples; rather, the examples are intended to illustrate only one of many possible embodiments. Further, the same reference numbers in different drawings can identify the same or similar elements.

1. A method comprising:
receiving, by one or more processors, an image frame and an identification of a region of plaque in the image frame, wherein the image frame is taken by an imaging device while the imaging device was in a lumen depicted in the image frame;
identifying, by the one or more processors, a lumen-center of the lumen depicted in the image frame; and
generating, by the one or more processors and using at least the lumen-center, a lumen-centered arc having a coverage angle centered on the lumen-center.

2. The method of claim 1, wherein the identification of the region of plaque comprises a mask, wherein a region of pixels in the mask corresponds to the region of plaque depicted in the image frame.

3. The method of claim 1, further comprising identifying endpoints of the region of plaque through which lines tangential to the region of plaque pass through the endpoints and the lumen-center, wherein the lines tangential to the region of plaque define the coverage angle of the lumen-centered arc.

4. The method of claim 1,
wherein the identification of the region of plaque comprises a mask, wherein a region of pixels in the mask corresponds to the region of plaque in the image frame,
wherein the identification comprises data defining the positions of pixels in the mask corresponding to the region of plaque and expressed as polar coordinates relative to a device-center of the imaging device while the imaging device was in the lumen, and
wherein the method further comprises converting the positions of pixels from polar coordinates relative to the device-center, to polar coordinates relative to the lumen-center.

5. The method of claim 1, wherein identifying the lumen-center comprises:
computing a lumen contour of the lumen;
generating a spline estimate of the lumen contour; and
identifying the lumen-center as an estimation of a centroid from a plurality of samples of the spline estimate.

6. The method of claim 1,
wherein the image frame is a first image frame and is one of a plurality of image frames in a sequence,
wherein the lumen-center is a first lumen-center, and
wherein the method further comprises:
identifying a respective lumen-center for each of the plurality of image frames in the sequence;
applying a low-pass smoothing filter to the respective lumen-center for each of the plurality of image frames, including the first image frame and the first lumen-center; and
generating, using at least the lumen-center after applying the low-pass smoothing filter and endpoints of the device-centered arc, a second lumen-centered arc having a coverage angle centered on the smoothed lumen-center.

7. The method of claim 6, wherein applying the low-pass smoothing filter further comprises:
applying the low-pass smoothing filter over an array of lumen-centers for the plurality of images that is symmetric around the lumen-center of the image frame in the middle of the sequence, wherein the filter further comprises one or more filter coefficients that are applied on a first image frame and depend at least on the relative frame index between the image frame in the middle of the sequence and the first image frame.

8. The method of claim 6, wherein identifying the respective lumen-center for each of the plurality of image frames in the sequence further comprises:
identifying one or more image frames depicting a respective side branch off of the lumen; and
interpolating, for each of the one or more image frames, a respective lumen-center from lumen-centers of neighboring image frames in the sequence that do not depict the respective side branch.

9. The method of any claim 1, wherein the method further comprises:
generating, by the one or more processors and at least partially using the coverage angle of the lumen-centered arc, one or more calcium scoring metric values corresponding to the region of plaque.

10. The method of claim 1,
wherein the region of plaque is a first region of plaque of a plurality of regions of plaque in the image frame, and
wherein the method further comprises generating, for each of the plurality of regions of media, a respective lumen-centered arc having a respective coverage angle centered on the lumen-center.

11. A system comprising:
one or more processors configured to:
receive an image frame and an identification of a region of plaque in the image frame, wherein the image frame is taken by an imaging device while the imaging device was in a lumen depicted in the image frame;
identify a lumen-center of the lumen depicted in the image frame; and
generate, using at least the lumen-center, a lumen-centered arc having a coverage angle centered on the lumen-center.

12. The system of claim 11, wherein the identification of the region of plaque comprises a mask, wherein a region of pixels in the mask corresponds to the region of plaque depicted in the image frame.

13. The system of claim 11, wherein the one or more processors are further configured to:
identify endpoints of the region of plaque through which lines tangential to the region of plaque pass through the endpoints and the lumen-center, wherein the lines tangential to the region of plaque define the coverage angle of the lumen-centered arc.

14. The system of claim 11,
wherein the identification of the region of plaque comprises a mask, wherein a region of pixels in the mask corresponds to the region of plaque in the image frame,
wherein the identification comprises data defining the positions of pixels in the mask corresponding to the region of plaque and expressed as polar coordinates relative to a device-center of the imaging device while the imaging device was in the lumen, and
wherein the one or more processors are further configured to convert the positions of pixels from polar coordinates relative to the device-center, to polar coordinates relative to the lumen-center.

15. The system of claim 2214, wherein converting the positions of the pixels further comprises:
converting the positions of the pixels from polar coordinates relative to the device-center to Cartesian coordinates; and
converting the positions of the pixels from Cartesian coordinates to polar coordinates relative to the lumen-center.

16. The system of claim 11, wherein to identify the lumen-center, the one or more processors are further configured to:
compute a lumen contour of the lumen;
generate a spline estimate of the lumen contour; and
identify the lumen-center as an estimation of a centroid from a plurality of samples of the spline estimate.

17. The system of claim 11
wherein the image frame is a first image frame and is one of a plurality of image frames in a sequence,
wherein the lumen-center is a first lumen-center, and
wherein the one or more processors are further configured to:
identify a respective lumen-center for each of the plurality of image frames in the sequence;
apply a low-pass smoothing filter to the respective lumen-center for each of the plurality of image frames, including the first image frame and the first lumen-center; and
generate, using at least the lumen-center after applying the low-pass smoothing filter and endpoints of the device-centered arc, a second lumen-centered arc having a coverage angle centered on the smoothed lumen-center.

18. The system of claim 11, wherein the one or more processors are further configured to:
display on a display coupled to the one or more processors the image frame annotated with the lumen-centered arc.

19. The system of claim 18, wherein to display the image frame annotated with the lumen-centered arc, the one or more processors are further configured to:
display the image frame within a field of view of a display viewport having a boundary, including displaying the lumen-centered arc along the boundary of the display viewport.

20. One or more non-transitory computer-readable storage media storing instructions that when executed by one or more processors, causes the one or more processors to perform operations comprising:
receiving, by one or more processors, an image frame and an identification of a region of plaque in the image frame, wherein the image frame is taken by an imaging device while the imaging device was in a lumen depicted in the image frame;
identifying, by the one or more processors, a lumen-center of the lumen depicted in the image frame; and
generating, by the one or more processors and using at least the lumen-center, a lumen-centered arc having a coverage angle centered on the lumen-center.
